(19) United States
(12) Patent Application Publication
(10) Pub. No.: US 2021/0085131 A1
(43) Pub. Date: Mar. 25, 2021

(54) Reusable pad for exfoliating the skin

(71) Applicant: Marcelo Osvaldo Raidán, Quilmes (AR)

(72) Inventor: Marcelo Osvaldo Raidán, Quilmes (AR)

(21) Appl. No.: 16/933,213

(22) Filed: Jul. 20, 2020

Related U.S. Application Data
(60) Provisional application No. 62/905,596, filed on Sep. 25, 2019.

(51) Int. Cl. A47K 7/02; A61K 8/02

(52) U.S. Cl. CPC A47K 7/02; A45D 2200/1054; A61K 2800/28; A61K 8/0208

(57) ABSTRACT

A water-dissolvable and environmentally friendly pad for exfoliating the skin is obtained by mixing baking soda, powdered Foam Glass and PVA-co-VAC. Its simple manufacturing process, performed entirely at room temperature, includes mixing the powders, adding the glue, pouring the mixture into molds and letting it dry by evaporation. Due to its characteristics, the resulting pad is reusable and can be rinsed, not losing its abrasive properties as well as its ability to clean grease, remove dead skin and clean, alter use. It has a spongy, moist, elastic, flexible and resistant structure, not toxic to the skin, nor can it be absorbed by it.

### CROSS-REFERENCE TO RELATED APPLICATION

This application claims the benefit of U.S. Provisional Patent Application No. 62/905,596, .filed Sep. 25, 2019, which is incorporated by reference herein in its entirety.

### TECHNICAL FIELD

This invention relates generally to the field of cosmetic articles and, more particularly, to articles for exfoliating the skin, comprising a water-soluble support and exfoliating particles carried by the support, and methods thereof notably in the cosmetic field for gentle exfoliation of the skin.

### BACKGROUND OF THE INVENTION

The cosmetic benefits of gentle exfoliation of the skin, and particularly the skin of the face, are well known in the field. For this reason, a wide variety of products for exfoliating Me skin has been developed. These products range from multiple-use articles (having mostly solid support) such as sponges, bath and shower mitten, and pumice stones to single-use products generally having liquid or viscous support such as lotions, creams and scrubs. Each of these products have different levels of abrasive power which often derives from the implementation of micro-particles in them. While some of them are too soft and no tangible effect is achieved by their use, others are too hard and may cause redness and discomfort in sensitive skins. An article that achieves a desirable balance between effectiveness and gentleness, achieving high, quality exfoliating results for different skin types, would doubtlessly be an advancement to the field. Even more so, if this is achieved through an easy and affordable manufacturing method and with little to no environmental impact.

In this framework, the use of polymers, and in particular of water-soluble polymers as support for the exfoliating particles, has proved to provide some advantages with respect to other products and methods for exfoliating the skin. However, none of the products nowadays available to the public are able to offer a composition which is effective for exfoliating by itself without the need of applying any additional product such as soaps, lotions or creams, while at the same time having a spongy and flexible structure, and being resistant enough to be reused multiple times before discarding. The long time that the exfoliating of the skin takes, the additional products that are needed for achieving an acceptable result, and the economic and environmental costs that their use imply, are some of the defects in the field of cosmetic exfoliating that an eco-friendly, affordable, easy to manufacture and easy to use cosmetic article like the one herein disclosed would come to solve, advancing the field.

### SUMMARY OF THE INVENTION

The main object of the present invention is to provide a new cosmetic article for exfoliating the skin, in the form of a reusable water-dissolvable and environmentally friendly exfoliating pad. A pad created in accordance with the present invention possesses a spongy and flexible structure that allows it to effectively exfoliate the skin without causing redness or discomfort, and without the need of applying any additional cleansing product, just by moistening the skin with water, exfoliating the skin with the product and rinsing.

The composition of this pad was formulated taking advantage of the benefits of three well-known components, and of the unpredicted synergetic advantages of using them together in the proportions here disclosed and through the methods here explained. These three products are PVA-co-VAC, (commonly sold as transparent school glue), which is water-soluble and acts like support for the micro-particles, Sodium Bicarbonate (baking soda), used as a builder and to achieve the desired texture, moisture, density and durability, and Foam Glass as an exfoliating agent. The Foam Glass has a surprisingly effective affinity with the baking soda, and when used together with the PVA-co-VAC as hereby prescribed they bind together forming a uniform, homogeneous and resistant structure providing a cosmetic article of unique characteristics and great exfoliating performance. The proportions for the use of these ingredients may vary depending on the viscosity of the glue and the desired final result, admitting a wide spectrum of varieties being their limits as follows: PVA-co-VAC 30% to 70%, Baking Soda 5% to 65%, and Foam Glass 5% to 65%. In the preferred embodiment proportions are PVA-co-VAC 40%, Baking Soda 30%, and Foam Glass 30%. Other embodiments further include additional ingredients including but not limited to perfumes and colorants. This micro abrasive structure allows the function of scraping, cleaning and stimulating the epidermis to generate new tissue. (Cleansing excess fat and accumulated dead cells)

An exfoliating pad in accordance with the present invention, is achieved by combining some or all of the following aspects:
(a) The support material, used in a 30% to 70% proportion, is a water-solvable polymer, preferably poly (vinyl alcohol-co-vinyl acetate) (“PVA-co-VAC”; chemical composition: CH2CHOH-co-(C4H6O2)n) in the form of clear school glue, but it may be replaced by other PVAc or PVOH variants as long as the utility is conserved.
(b) The builder, used in a 5% to 65% proportion, is an alkali salt, sodium bicarbonate (chemical composition: NaHCO3) but it may be replaced by other builders as long as the utility is conserved.
(c) The exfoliating particles, used in a 5% to 65% proportion, are micro-particles having a grain size of no more than 250 μm of diameter, preferably powdered Foam Glass or cellular glass (such as Owens Corning's “FOAMGLAS®” or the cellular glass sold by Ningbo Yoyo Foam Glass Co, Ltd.) but they could be replaced by other microparticles as long as the utility is conserved.
(d) The support material, the builder and the exfoliating particles are thoroughly mixed at room temperature and the mixture is poured into melds having a thickness of between 0.5 mm and 5 mm, being 2 mm the optimum thickness that allows an optimal mechanical strength and flexibility for handling and 1.5 mm to 2.5 mm the ideal range for an improved cost-benefit ratio.
(e) The molds can be rounded, squared, or have different shapes, including geometric shapes and decorative shapes such as, for example, leaves, hearts or stars.
(f) The pad has its own humidity that lasts for quite some time (depending on environmental conditions), since when the bicarbonate reacts, it generates H2O. keeping it moist.
(g) It is reusable and can be rinsed, not losing its abrasive properties as well as its ability to clean grease, removing dead skin and cleaning, after use.
(h) Its humidity can last up to two or three months, depending on the storage conditions, as it dries slowly.

Furthermore, a pad created in accordance with the present invention offers additional advantages when compared to its predecessors, these advantages including but not limited to: (a) It has a spongy, moist, elastic, flexible and resistant structure, (b) it is not toxic to the skin, nor can it be absorbed by it, (c) it is soluble in water, (d) it does not form mold, or any type of fungus, (e) it is biodegradable, (f) its texture makes it possible to feel the abrasive micro particles that will exfoliate the epidermis mechanically, that relieved by its elasticity and moisture, make it pleasant to the touch, (g) its characteristics reduce the time of the treatment that is needed to clean the skin when compared to alternative products.

A method for combining the above-mentioned ingredients in the above-mentioned proportions for obtaining a water-dissolvable exfoliating pad in accordance with the present invention is also disclosed, comprising the steps of:

(1) Exhaustively mixing the Raking Soda and the Foam Glass in the appropriate proportions for about 15 minutes.
(2) Adding the PVA-co-VAC to the mixture of step 1, at room temperature (25 degrees Celsius±5 degrees Celsius) and mixing it, together until obtaining a homogeneous mixture.
(3) Introducing the mixture of step 2 into a mold having the desired shape for the resulting pad. and a thickness of between 0.5 millimeters and 5 millimeters.
(4) Letting it dry by evaporation at room temperature for 24 to 48 hours.

The appropriate use of an exfoliating pad in accordance with the present invention, to achieve the desired benefits without reddening the skin consists of three simple steps:

1—Wet the skin well with steam (2 to 3 minutes) or with warm water (1 to 2 minutes); 2—Apply the pad in a gentle circular way through the area to be treated for approximately 1 minute; 3—Rinse with plenty of water and repeat the process if necessary. This process should be repeated once a week for dry skin. For sensitive skin, it should be done every ten days previously testing it in a small, little visible, area of the face, for example, behind the ears. Acne-free oily skin can repeat it once or twice a week. Skin with acne should not do it. The recommended areas for the application of the product are: The forehead, the nose, the chin and behind the ears. It is also recommended for the area of the elbows, feet, and other parts of the body, without problems.

### BRIEF DESCRIPTION OF THE DRAWINGS

FIG. 1 is a schematic front view of the first step of a process to manufacture an embodiment of a reusable exfoliating pad in accordance with the present invention, consisting of preparing the powder mixture.

FIG. 2 is a schematic front view of the second step of a process to manufacture an embodiment, of a reusable exfoliating pad in accordance with the present invention, consisting of mixing the powders.

FIG. 3 is a schematic front view of the third step of a process to manufacture an embodiment of a reusable exfoliating pad in accordance with the present invention, consisting of adding the glue.

FIG. 4 is a schematic front view of the fourth step of a process to manufacture an embodiment of a reusable exfoliating pad in accordance with the present invention, consisting of mixing the powders and the glue.

FIG. 5 is a schematic front view of the fifth step of a process to manufacture an embodiment of a reusable exfoliating pad in accordance with the present invention, consisting of pouring the mixture into a mold.

FIG. 6 is a schematic front view of the sixth step of a process to manufacture an embodiment of a reusable exfoliating pad in accordance with the present invention, consisting of letting the mixture dry.

FIG. 7 is a perspective view of an embodiment of a reusable exfoliating pad in accordance with the present invention.

### DETAILED DESCRIPTION AND BEST MODE Of IMPLEMENTATION

Disclosed is a reusable cosmetic article for exfoliating the skin, particularly useful for exfoliating the skin of the face. This article comes in the form of a reusable pad or sponge which is water-dissolvable and has a has a spongy, moist, flexible and resistant structure. This is attained in a cost-efficient manner thanks to a simple manufacturing process comprising the mixture of two powders and a glue, mixture which is then poured in molds and dried to obtain the final product. A method for manufacturing the pads is also disclosed.

Some general aspects of the present invention have been summarized so far in this disclosure. Hereinafter, a detailed description of the invention as illustrated in the drawings will be provided. While some aspects of the invention will be described in connection with these drawings, it is to be understood that the disclosed embodiments are merely illustrative of the invention, which may be embodied in various forms. The specific, materials, methods, structures and functional details disclosed herein are not to be interpreted as limiting. Instead, the intended function of this disclosure is to exemplify some of the ways -including the presently preferred ways- in which the invention, as defined by the claims, can be enabled for a Person of Ordinary Skill in the Art. Therefore, the intent of the present disclosure is to cover all variations encompassed within the spirit and scope of the invention defined by the appended claims, and any reasonable equivalents thereof.

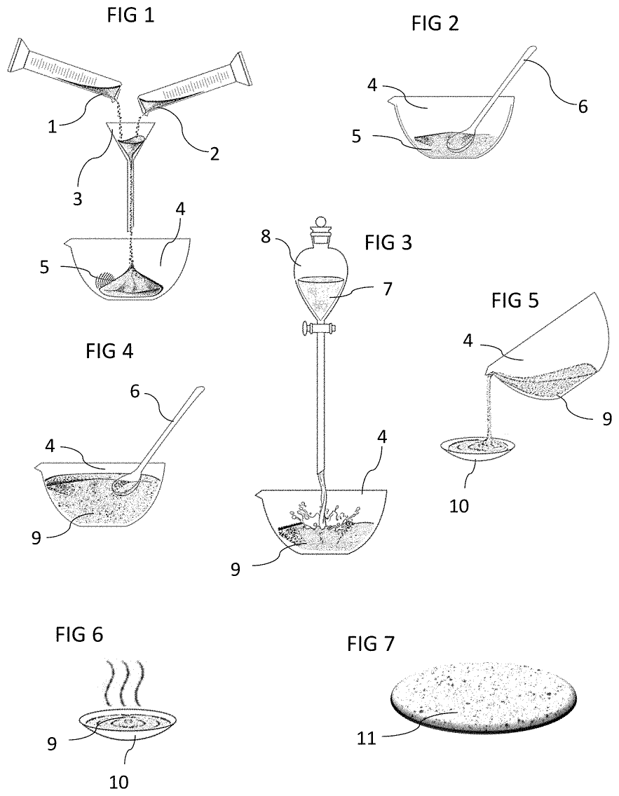

Referring to the drawings in more detail, FIG. 1 schematically illustrates the first step of the process in which the powders are poured together into a container 4, in the adequate proportions to form the powder this example the powders used are Baking Soda 1, and Foam Glass 2. A funnel 3 is used to facilitate the mixture in accurate proportions, but in other embodiments the use of the funnel can be omitted. The baking soda 1 is added in proportion such that it will account for 25% of the total volume of the final mixture. In other embodiments, other percentage may be used as long as the general proportions established by this specification are respected. The Foam Glass 2 is added in a proportion such that it will account for 25% of the total volume of the final mixture. In other embodiments, other exfoliating particles may be used instead of or in addition to Foam Glass as long as the utility is conserved, In the preferred embodiment, powdered Foam Glass or cellular glass is used, such as commercially available Owens Corning's “FOAMGLAS®” ®, “Ningbo Yoyo Foam Glass Go Ltd.” and others, which is sold in the form of “Insulating rubble” that, in order to be convened to the required powder form, can be placed in an industrial machine for brick dust or similar which produces a grain between 150 μm and 250 μm approximately. Alternatively, powdered Foam Glass can be manually obtained by rubbing block against block. The resulting mixture is called the powder mixture 5, which shall incorporate in this step any other powders in the embodiments that include additional granular elements and which in total amount, in the described embodiment, to 50% of the total volume of the final mixture. In other embodiments the powder mixture 5 may amount to other proportions ranging from 30% to 70% of the total volume of the final mixture. Different proportions yield different results. For example, 45% of baking soda and 5% Foam Glass powder, keeping the remaining 50% of glue, would form a solid but less abrasive product for the purpose of exfoliating more sensitive skin including the face. The glue may vary its percentage depending on its viscosity. Greater exfoliation power can be achieved by inverting the aforementioned percentages in 5% sodium bicarbonate and 45% Foam Glass powder and 50% glue. This will make a harder product, with more capacity for the exfoliation of very thick skin.

FIG. 2 shows how the powder mixture 5 in the container 4 is exhaustively mixed at room temperature until acquiring a homogeneous state. This can be achieved by hand with the use of a spoon 6 stirring vigorously for at least 15 minutes, or with the use of industrial or laboratory “V” powder mixers, or other types of powder mixers to achieve homogeneity.

In FIG. 3 the glue 7 is added to the container 4, so that together with the powder mixture 5 already in it, it forms the final mixture 9 completing the 100% of the final volume. This step can be implemented, in some embodiments, with the use of a separator funnel 8. As used in this specification and the appended claims, the term “glue” refers to a water-solvable polymer, preferably poly (vinyl alcohol-co-vinyl acetate) (“PVA-co-VAC”; chemical composition: CH2CHOH-co-(C4H6O2)n), but it may be replaced by other PVAc (PVAc or PVOH) variants or even other polymers as long as the utility is conserved. PVA-co-VAC is a colorless liquid with a distinctive aroma, it is a polyvinyl acetate, a polymer widely used in the industry. This glue dissolves in water at different temperatures depending on the brands. It is washable and does not stain.

In the preferred embodiment, the type of glue used is Transparent liquid adhesive poly (vinyl alcohol-co-vinyl acetate) school glue, (Voligoma®, Akapol SA, Argentina, with a with a molecular weight of 47,000 g/mol and 12% vinyl acetate). Due to its viscosity, it is one of those glues recommended to comply with drying times and product structure, (24 hours/30 hours between 22° C./25° C. with 40% to 50% ambient humidity, summer season or laboratory controlled). Other brands and varieties have also been tested, whose contents have different viscosity and water dispersion. The pads can still be manufactured with these products, but it is observed that an excessive water proportion, in comparison to the same quantity in grams in other brands such as “Voligoma”, have a negative impact in the product's drying time, resistance and elasticity. However, these and other brands may still be used, provided that the glue is based on polyvinyl alcohol, (PVOH) and the drying times and proportions are adjusted to achieve an acceptable final result. Therefore, the result is not limited to a specific brand.

FIG. 4 shows how the final mixture 9 in the container 4 is exhaustively mixed at room temperature until acquiring a homogeneous state. This can be achieved by hand with the use of a spoon 6 stirring vigorously for at least 15 minutes, or with the use of industrial or laboratory mixers, or other types of mixers to achieve homogeneity. The resulting final mixture 9 shall incorporate in this step any other liquid or viscous ingredients such as perfumes and colorants in those embodiments that include them.

In FIG. 5, the final mixture 9 is poured into molds 10, having a thickness of between 0.5 mm and 5 mm, being 2 mm the optimum thickness that allows an optimal mechanical strength and flexibility for handling and 2 mm to 3 mm the ideal range for an improved cost-benefit ratio. The molds can be rounded, squared, of have different shapes, including geometric shapes and decorative shapes such as leaves, hearts or stars. In FIG. 6 the final mixture 9 in the molds 10 are dried naturally by evaporation at room temperature for 24 to 48 hours. Once the mixture is dried, the pads 11 (FIG. 7) are extracted from the molds 10.

The description as set forth is not intended to be exhaustive or to limit the invention to the precise form disclosed. Many modifications and variations are possible in light of the teachings above without departing from the spirit and scope of the forthcoming claims.

1. A reusable water-dissolvable exfoliating pad for the skin comprising 5% to 65% in volume of baking soda, 5% to 65% in volume of powdered Foam Glass and 30% to 70% in volume of a water solvable polymer.

2. The reusable water-dissolvable exfoliating pad for the skin of claim 1 wherein said water solvable polymer is PVA-co-VAC.

3. The reusable water-dissolvable exfoliating pad for the skin of claim 1 wherein said water solvable polymer is a Transparent liquid adhesive poly (vinyl alcohol-co-vinyl acetate) school glue.

4. A reusable water-dissolvable exfoliating pad for the skin comprising 30% in volume of baking soda, 30% in volume of powdered Foam Glass and 40% in volume of a water solvable polymer.

5. The reusable water-dissolvable exfoliating pad for the skin of claim 4 wherein said water solvable polymer is PVA-co-VAC.

6. The reusable water-dissolvable exfoliating pad for the skin of claim 4 wherein said water solvable polymer is a Transparent liquid adhesive poly (vinyl alcohol-co-vinyl acetate) school glue.

7. A method for manufacturing a water-dissolvable exfoliating pad for the skin comprising the steps of (a) exhaustively mixing 5% to 65% in volume of Baking Soda and 5% to 65% in volume of powdered Foam Glass for about 15 minutes to obtain a powder mixture, (b) adding 30% to 70% in volume of Transparent liquid adhesive poly (vinyl alcohol-co-vinyl acetate) school glue to the powder mixture obtained in step a, at room temperature and mixing it together until obtaining a homogeneous final mixture, (c) introducing the final mixture obtained in step b into a mold having a thickness of between 1.5 millimeters and 2.5 millimeters, and (d) letting it dry in the mold by evaporation at room temperature for 24 to 48 hours.
